(19) United States
(12) Patent Application Publication
(10) Pub. No.: US 2023/0113963 A1
(43) Pub. Date: Apr. 13, 2023

(54) NEUROMUSCULAR TRANSMISSION MONITORING SYSTEM AND KINEMYOGRAPHY SENSOR

(71) Applicant: GE Precision Healthcare LLC, Wauwatosa, WI (US)

(72) Inventor: Jami Matti Jormakka, Espoo (FI)

(73) Assignee: GE Precision Healthcare LLC, Wauwatosa, WI (US)

(21) Appl. No.: 17/496,898

(22) Filed: Oct. 8, 2021

(51) Int. Cl. A61B 5/11; A61B 5/00; A61B 5/395; A61B 5/388; A61B 5/30

(52) U.S. Cl. CPC A61B 5/1114; A61B 5/6826; A61B 5/395; A61B 5/388; A61B 5/303; A61B 2562/0261

(57) ABSTRACT

A kinemyography sensor includes a support frame and a flexible substrate, wherein at least a portion of the flexible substrate is attached to the support frame. The support frame is configured to attach to a patient's thumb and forefinger and has a bendable middle section configured to bend in response to movement of the patient's thumb. A printed stimulation circuit is printed on the substrate and includes a pair of stimulation electrodes configured to adhere to a patient's skin to deliver a kinemyography stimulus, and a printed bend sensor is printed on the substrate and located on the bendable middle section of the support frame, wherein the printed bend sensor is configured to sense the bending of the support frame.

### BACKGROUND

The present disclosure generally relates to neuromuscular monitoring of patients, and more particularly to a kinemyography sensor and monitoring system.

Neuromuscular transmission (NMT) is the transfer of an impulse between a nerve and a muscle at the neuromuscular junction. An NMT may be blocked in a patient, such as a patient undergoing a surgical procedure by neuromuscular blocking agents/drugs. Neuromuscular blocking agents cause transient muscle paralysis and prevent the patient from moving spontaneously.

It is often desirable to monitor the level of neuromuscular block in a patient to ensure that appropriate block is provided for a given procedure and also to limit the amount of neuromuscular blocking agent administered to a patient to the minimum amount needed to achieve the desired level of paralysis. Patient monitoring systems, and specifically neuromuscular transmission monitoring systems, are utilized to determine a patient's muscle response, and thus the level of neuromuscular block experienced by a patient. Several types of neuromuscular transmission (NMT) monitoring systems are available, including electromyography systems, kinemyography systems, and acceleromyography systems, to name a few. NMT monitors utilize an electrical stimulus provided to a patient's motor nerve and measure a muscle response thereto. Typically, the stimulus is provided to a patient's ulnar nerve near the wrist and the response of the muscle near the thumb, the adductor pollicis, is monitored. The evoked muscle responses are monitored via any of several methods listed above. In kinemyography, the degree of distortion, or bending, of the sensor due to the muscle response, such as at the patient's thumb, is measured.

In clinical settings, the nerve stimulator is often attached to a patient (e.g., on the patient's skin above the ulnar nerve) and an electrical stimulation current is applied to the patient before induction of the anesthesia or immediately thereafter. Thereby, a baseline value response is recorded by the NMT monitor and used to normalize the muscle response once the muscle relaxant is administered. Evoked muscle responses are then monitored, such as throughout the surgical procedure, to determine the patient's level of neuromuscular blockage.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one embodiment, a kinemyography sensor includes a support frame and a flexible substrate, wherein at least a portion of the flexible substrate is attached to the support frame. The support frame is configured to attach to a patient's thumb and forefinger and has a bendable middle section configured to bend in response to movement of the patient's thumb. A printed stimulation circuit is printed on the substrate and includes a pair of stimulation electrodes configured to adhere to a patient's skin to deliver a kinemyography stimulus, and a printed bend sensor is printed on the substrate and located on the bendable middle section of the support frame, wherein the printed bend sensor is configured to sense the bending of the support frame.

In one embodiment, a kinemyography sensor includes a flexible substrate having a stimulation section, a sensor section, and a connection section. The stimulation section has a pair of stimulation electrodes printed thereon and configured to adhere to a patient's skin to deliver a kinemyography stimulus. The sensor section has a printed bend sensor printed thereon, wherein the printed bend sensor is configured to be positioned between a patient's thumb and forefinger to sense movement of the patient's thumb. The connection section is at a first end of the substrate, the connection section having a plurality of contact pads printed thereon and configured to mate with a sensor connector of a neuromuscular transmission monitoring device.

In one embodiment, a neuromuscular transmission monitoring system includes a kinemyography sensor and a neuromuscular transmission monitoring device having a sensor connector configured to removably mate with a first end of the kinemyography sensor so as to receive sensing signals therefrom. The kinemyography sensor includes a flexible substrate having a stimulation section, a sensor section, and a connection section. The stimulation section has a pair of stimulation electrodes printed thereon and configured to adhere to a patient's skin to deliver a kinemyography stimulus. The sensor section has a printed bend sensor printed thereon and configured to sense movement of the patient's thumb in response to the stimulus, wherein the printed bend sensor is a resistive sensor or a piezoelectric sensor. The connection section is at the first end of the substrate and is configured to electrically connect to the sensor connector.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 shows an exemplary neuromuscular transmission monitoring system.

FIG. 2 shows a bottom view of the kinemyography sensor of FIG. 1.

FIG. 3 shows a cross sectional view of a stimulation section of an exemplary kinemyography sensor.

FIG. 4 depicts one embodiment of a flexible substrate for a kinemyography sensor with a printed stimulation circuit and a printed bend sensor thereon.

FIG. 5 depicts another embodiment of a flexible substrate for a kinemyography sensor with a printed stimulation circuit and a printed bend sensor thereon.

FIGS. 6 and 7 depict cross sectional views of exemplary embodiments of printed bend sensors according to the present disclosure.

### DETAILED DESCRIPTION

Kinemyography (KMT) measures a muscle response of a patient based on the amount of distortion or bending induced by a patient's muscle response on a bend sensor. A bend sensor may be placed between the thumb and forefinger on a patient where the electrical stimulation is delivered to a patient's ulnar nerve or a patient's median nerve at the patient's wrist. Current kinemyography sensors are reusable sensors that are used with multiple patients over a relatively long service life.

The inventor has recognized that current reusable kinemyography sensors are problematic for several reasons. First, they pose a contamination risk due to their use by multiple patients. Further, reusable sensors are prone to breakage and sensing accuracy degradation over their long service life, sometimes breaking or malfunctioning in undetectable ways resulting in undetected inaccuracies in the sensing output.

In view of the foregoing problems and challenges in the relevant art, the inventor has developed the disclosed single-use kinemyography sensor that can be manufactured for relatively low cost, provides an intuitive form factor, and yields reliable and replicatable measurements. The disclosed sensor is a printed kinemyography sensor wherein the stimulation circuit and the bend sensor are both incorporated onto a single flexible substrate to be attached to the patient's hand and wrist. The disclosed printed kinemyography sensor utilizes screen printing or other flexible printing techniques and enables printing of both the stimulation circuit and the sensing circuit in one process step, or in some embodiments in only a few process steps depending on the type of bend sensor utilized. The single-piece kinemyography sensor minimizes opportunities for assembly mistakes and damage during transport and is easy and intuitive to apply to the patient.

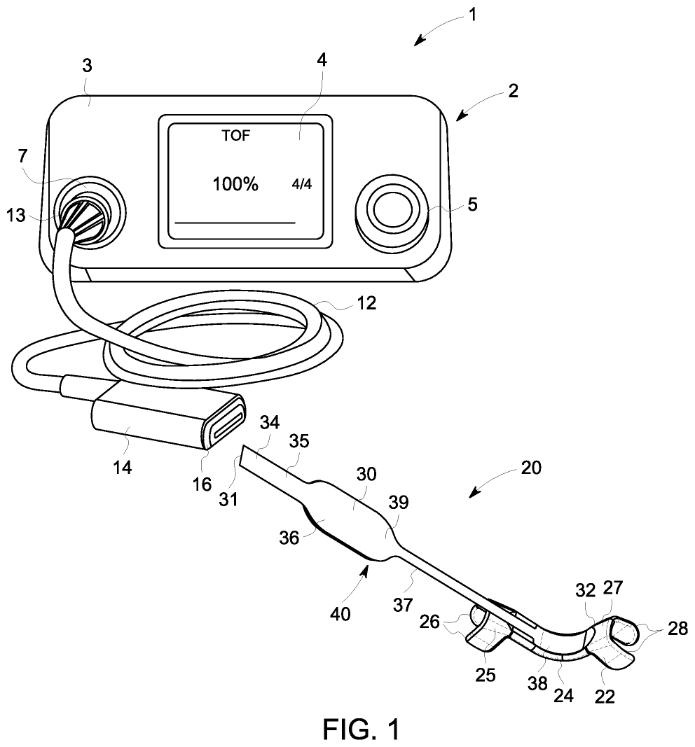

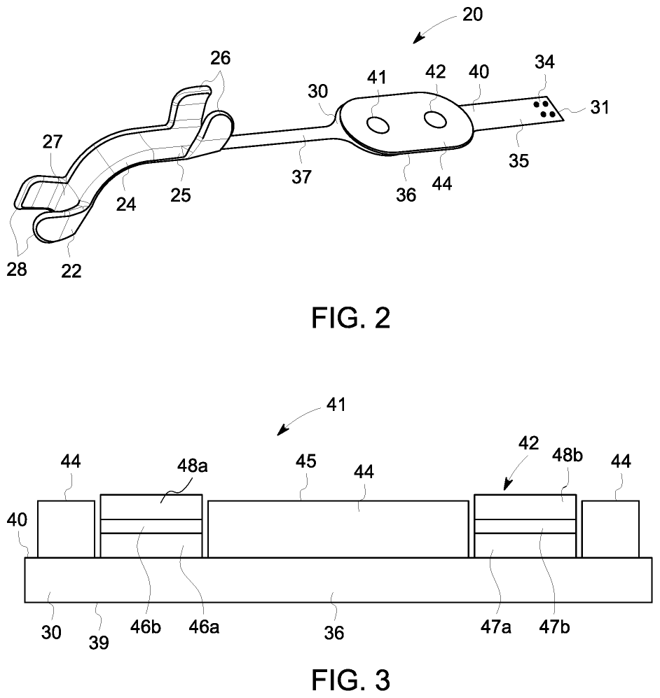

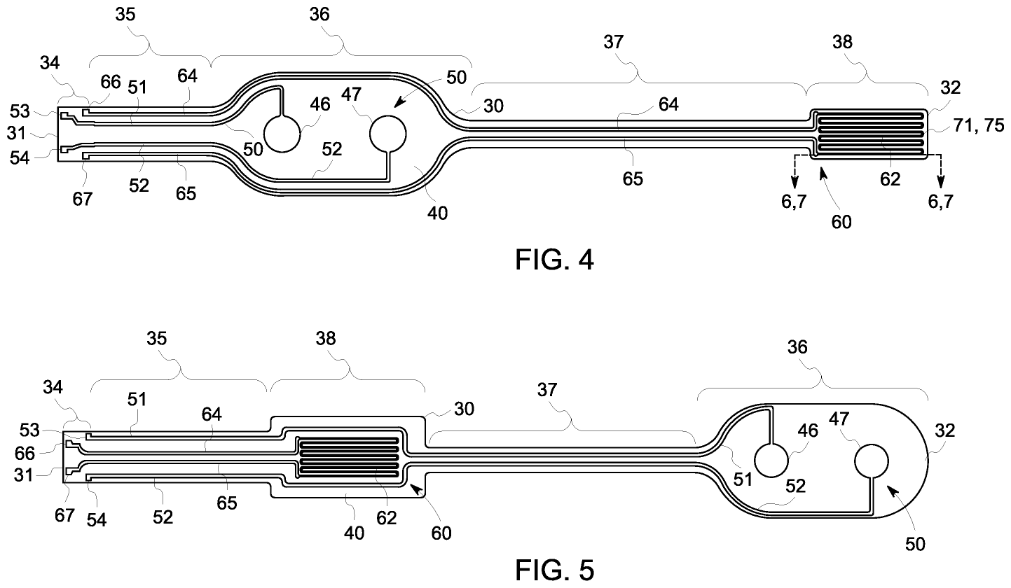

FIGS. 1-7 depict exemplary embodiments of a printable single-use kinemyography sensor. In FIG. 1, an exemplary kinemyography sensor 20 is depicted in conjunction with an NMT monitoring device 2, together forming an NMT monitoring system 1. The printed kinemyography sensor 20 is printed on a flexible substrate 30 forming a single elongated piece having a first end 31 and a second end 32. The elongated flexible substrate 30 has a stimulation section 36 on which a pair of stimulation electrodes are printed and a sensor section 38 on which a bend sensor is printed. (see FIGS. 4-7) The flexible substrate 30 comprises a flexible material, such as a polyethylene terephthalate (PET), having a topside 39 and a bottom side 40 on which stimulation circuit 50 and a sensing circuit 60 are printed. FIG. 1 shows a top view of the printed kinemyography sensor where the topside 39 is visible, and FIG. 2 (and FIGS. 4 and 5) is a bottom view thereof showing the bottom side 40 in various configurations.

The printed kinemyography sensor 20 further includes a connection section 34 adjacent to the first end 31 of the substrate that is configured to mate with and electrically connect to a sensor connector 14 of the NMT monitoring device 2. More particularly, the connection section 34 includes multiple printed contact pads that are configured to electrically connect to corresponding contacts in the sensor connector 14 of the NMT monitoring device 2. The connection section 34 is configured to mate with the connection port 16 of the sensor connector 14, which in the depicted embodiment is performed by sliding the connection section 35 at the first end 31 of the flexible substrate 30 into the sensor connection port 16 of the sensor connector 14. Thus, the connection port 16 is configured to receive the connection section 35. In other embodiments the connection section 35 may include a connector, such as a non-printed male or female connection end, that is attached to the substrate 30 and configured to mate with the sensor connector 14.

The sensor connector 14 is at the end of a cable 12. At the opposing end of the cable 12 is a device end 13 that connects to the NMT monitoring device 2. The NMT monitoring device 2 includes a housing 3 with a sensor port 7 configured to mate with the device end 13 of the cable 12. In the depicted embodiment, the housing 3 also holds a display 4 and a user input element 5. The user input element 5 may be configured to allow a user to control function of the NMT monitoring system 1, including to initiate a measurement on the patient and/or to control a mode of the monitoring device 2, such as to instruct automatic periodic NMT measurement on the patient.

The NMT monitoring device 2 is configured to process the electrical signals received from the kinemyography sensor 20 and to determine a level of neuromuscular blockage for the patient. In one embodiment, the NMT monitoring device 2 is configured to determine a train of four (TOF) of the patient. The measured and determined level of neuromuscular blockade may be displayed on the display 4, which in the depicted example is displayed as a number of detected muscle responses for forced stimulation and as a percentage.

The kinemyography sensor 20 shown in FIGS. 1 and 2 includes a support frame 22 connected to the flexible substrate 30. The support frame 22 is configured to attach to a patient's thumb and forefinger and has a bendable middle section 24 configured to bend in response to movement of the patients thumb following stimulation of the patient's motor nerve by the stimulation circuit 50 on the sensor 20. For example, the support frame 22 may be a molded polymer, foam or plastic material that is lightweight and flexible such that it conforms to the patient's hand and moves in response to the muscle action of the patient but is also sufficiently rigid to support the sensor and to direct movement of the thumb and forefinger for reliable measurement. The frame may be molded from flexible elastomer or foamed plastic, for example.

The support frame 22 may be a curved shape piece, such as having a first leg 25 configured to attach to the patient's thumb and a second leg 27 configured to attach to the patient's forefinger. In the depicted example, the support frame 22 is attached to the patients thumb and forefinger by finger prongs 26 and 28. Specifically, the first leg 25 has a first set of finger prongs 26 configured to clasp or wrap around a patient's thumb. The second leg 27 has a set of finger prongs 28 configured to clasp or wrap around the patient's forefinger. Thereby, the support frame 22 is held in place on the patient's hand.

The flexible substrate 30 is attached to the support frame 22 such that the printed bend sensor 62 is located on the bendable middle section 24 of the support frame 22. More particularly, the sensor section 38 on which the printed bend sensor 32 is mounted is attached to the middle section 24 of the support frame 22, such as adhered thereto. The sensor 38 may be attached to the bendable middle section 24 with an adhesive, such as double-sided pressure-sensitive adhesive tape with acrylic adhesive. The adhesive is located between the support frame and flexible substrate as to not be exposed to user and may be applied over the entirety of the sensor section 38 including over the bend sensor 62, for example, or may be applied around the edges of the sensor section 38, such as to avoid the area of the printed bend sensor 62.

Referring also to FIGS. 4 and 5, the flexible substrate 30 of the sensor 20 is a single elongated piece having a first end 31 and a second end 32. A connection section 34 is located at the first end 31 and configured to mate with the sensor connector 14 as described above. The elongated body of the flexible substrate 30 further includes a first lead section 35 with leadwires printed thereon, including stimulation leadwires 51 and 52 that connect to the pair of stimulation electrodes and sensor leadwires 64 and 65 that connect to the printed bend sensor 62. The elongated body of the flexible substrate 30 further includes a stimulation section 36 on which the stimulation electrodes 41 and 42 (including electrode pads 46 and 47) are printed, and a sensor section 38 on which the printed bend sensor 62 is printed. A second lead section 37 is positioned and connects between the stimulation section 36 and the sensor section 38. The second lead section 37 includes at least one set of leadwires, which may be the stimulation leadwires 51 and 52 or the sensor leadwires 64 and 65, depending on the arrangement of the stimulation and sensing sections.

The stimulation section 36 and the sensor section 38 may be variously arranged on the elongated substrate 30. The shape of the elongated substrate 30 and the position of leadwires are adjusted accordingly, and various shapes and lead wire arrangements are within the scope of the present disclosure. FIG. 4 exemplifies an embodiment where the sensor section 38 is at the second end 32 of the flexible substrate—i.e., the first lead section 35 connects to the stimulation section 36 and the second lead section connects to the sensor section 38 and has the sensor leadwires 64 and 65 printed thereon. FIG. 5 depicts another embodiment where the stimulation section 36 is at the second end 32, and thus the first lead section 35 connects to the sensor section 38 and the second lead section 37 connects to the stimulation section 36 and has the stimulation leadwires 51 and 52 printed thereon.

In both embodiments, the shapes of each of the stimulation section 36 and the sensor section 38 may be adjusted as appropriate for a particular design and attachment to the patient. Similarly, the proportions and lengths of the first lead section 35 and the second lead section 37 may also be adjusted such that the stimulation section 36 is easily positionable on a patient's wrist and the sensor section 38, which is connected to the support frame 22, is comfortably positioned on a patients thumb and forefinger with enough slack that the patient can rotate their hand and wrist without undue restriction. The sensor may be sufficiently long and proportioned to accommodate a range of patients and various patient physiologies. In certain embodiments, multiple sensor sizes may be manufactured, and lengths and proportions of the various substrate sections 34-38 may be adjusted accordingly.

The simulation circuit 50 includes a pair of stimulation electrodes 41 and 42 and corresponding stimulation leadwires 51 and 51 and contact pads 53 and 54. Referring to FIGS. 2 and 3, each stimulation electrode 41, 42 comprises an electrode pad 46, 47 and an electrode gel layer 48a, 48b. As also shown in FIGS. 4 and 5, two electrode pads including first electrode pad 46 and second electrode pad 47 are printed on the bottom side 40 of the substrate 30, and particularly the stimulation section 36 of the substrate as described above. In the depicted example the stimulation circuit 50 includes a first electrode pad 46 connected to a first stimulation leadwire 51 that terminates at a first stimulation contact pad 53, and a second electrode pad 47 connected to a second stimulation leadwire 52 that terminates at a second stimulation contact pad 54. The first and second stimulation contact pads 53 and 54 located at the connection section 34 of the substrate 30 and exposed (see FIG. 2) such that they can contact corresponding connections within the sensor connector 14. In other embodiments the stimulation circuit may include additional electrodes and leadwires.

The elements of the stimulation circuit 50 are printed on the substrate 30 with a conductive ink, such as a silver-based conductive ink. In certain embodiments, the electrode pads 46, 47 may be printed with a silver/silver chloride (Ag/AgCl) ink that provides increase conductivity for delivering the stimulation current to the patient. In certain examples, the electrode pads 46, 47 may consist of two printed layers, including a first conductive ink and a second conductive ink. FIG. 3 illustrates one such example.

FIG. 3 shows a cross section of the stimulation section where the electrode pads 46, 47 each comprise two printed layers. A first electrode pad layer 46a, 47a may be comprised of a first conductive ink with low electric resistance, such as the same silver-based conductive ink used for printing the leadwires 51, 52 and contact pads 53, 54. A second electrode pad layer 46b, 47b may be printed on top of the first conductive ink and may be comprised of a second conductive ink with a different conductivity. In certain embodiments, the second conductive ink may be more conductive than first conductive ink. For instance, the first conductive ink may be a silver-based conductive ink and the second conductive ink may be a Ag/AgCl ink, which is used for the electrode pad of the circuit to provide lower electrode-skin interface impedance. In other embodiments, the electrode pads 46, 47 may only comprise a single layer of ink, which may be just the first conductive ink or just the second conductive ink.

A dielectric layer may be printed on top of the leadwire portions of the circuit 50, avoiding the electrode pads 46 and 47 and the contact pads 53 and 54, to isolate the circuit. Electrode gel is applied on top of the electrode pads 46 and 47. The first electrode gel pad 48a is applied over a first electrode pad 46 and a second electrode gel pad 48b is applied over the second electrode pad 47. In one embodiment, the electrode gel 48a, 48b may be printed on top of the respective electrode pads 46, 47.

An adhesive pad 44 is assembled onto the stimulation section 36 of the substrate 30, which may cover over at least a portion of the stimulation leadwires 51 and 52 but avoiding the stimulation electrodes 41 and 42. As shown in the figures, adhesive pad 44 is shaped to cover the stimulation section 36 of the substrate 30 and has two holes therein where each of the stimulation electrodes 41 and 42 are located. For example, the adhesive pad 44 may be a foam pad with adhesive on the bottom side 45 configured to adhere to a patient's skin. Thereby, the adhesive pad 44 attaches the electrodes 41 and 42 to the patient's skin. In certain embodiments, the thickness of the adhesive pad 44 is equal to or slightly less than the total thickness of the stimulation electrodes 41 and 42. Thus, the electrode gel 48a, 48b is flush with or slightly protruding from the bottom surface 45 of the adhesive pad 44.

Referring again to FIGS. 4 and 5, the sensor circuit 60 includes the printed bend sensor 62 and two printed sensor leadwires 64 and 65 extending from each end of the printed bend sensor 62 and terminating at a respective sensing contact pad 66, 67. The sensor leadwires 64 and 65 and the sensing contact pads 66 and 67 may be printed from the same conductive ink as the stimulation circuit 50, such as a silver-based ink as described above as the first conductive ink. The bend sensor 62 may be printed with the same ink as the rest of the sensing circuit 60. Alternatively, the bend sensor 62 may be printed utilizing a different ink and/or piezoelectric layer material, depending on the configuration of the bend sensor 62.

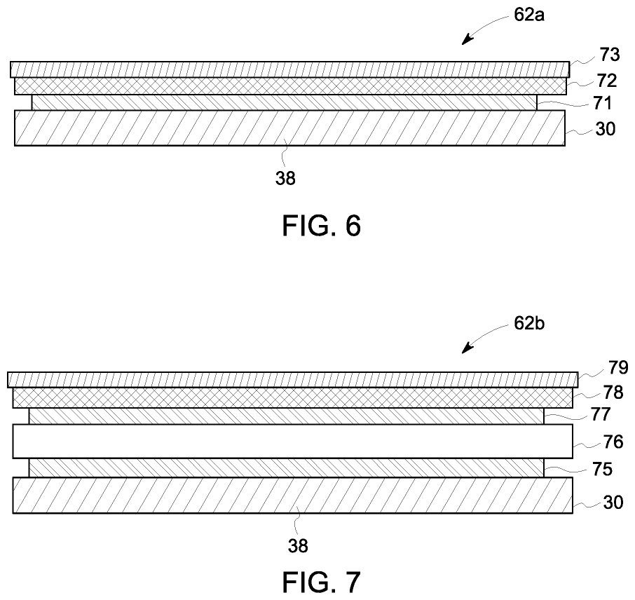

The bend sensor 62 is printed on the substrate 30, and specifically on the sensor section 38 thereof. The printed bend sensor is configured such that bending of the sensor section 38 in reaction to movement of the patient's thumb causes changes in the sensor that enable movement detection and/or measurement of the magnitude of movement. FIGS. 6 and 7 depict exemplary printed bend sensors 62, where FIG. 6 is a cross section of a printed resistive sensor 62a and FIG. 7 is cross section of an exemplary printed piezoelectric sensor 62b.

The resistive bend sensor 62a is configured to change resistance when bent so as to enable measurement of the movement of the patient's thumb. When attached to the support frame 22, the resistive sensor 62a bends with the bendable middle section 24 due to movement of the patient's thumb. As the resistive bend sensor 62a bends, the resistance progressively increases as the magnitude of the bend increases. FIG. 6 depicts an exemplary resistive bend sensor that includes a conductive ink layer 71, such as printed using the silver-based first conductive ink described above. A dielectric layer 72 may be printed on top of the conductive ink layer 71 to isolate and protect the conductive ink layer. In certain examples, the dielectric layer 72 may be printed over just the conductive ink layer 71, or may be printed or otherwise applied over the entire sensor section 38, and/or over the first and/or second lead sections 35 and 37 of the substrate 30.

As the resistive bend sensor 62a is bent due to movement of the patient's thumb, the cross section of the conductive ink layer 71 changes, thus changing the resistance. For the resistive embodiment, the NMT monitoring device 2 is configured to measure resistance across the resistive bend sensor 62a at predetermined intervals following a stimulation so as to measure the change in resistance a plurality of times throughout the resulting movement of the patient's thumb so as to measure a magnitude thereof.

Alternatively, the printed bend sensor 62 may include a printed piezoelectric bend sensor 62b configured to produce a charge when it is bent. Namely, deformation of the piezoelectric material in the sensor produces an electric charge resulting from the piezoelectric effect. This charge can be measured as an indicator of the bend magnitude, and thus the magnitude of movement and muscle response in the patient's thumb. The NMT monitoring device 2 receives and samples the charge a plurality of times throughout the resulting muscle response to as to measure a magnitude thereof.

In the embodiment depicted in FIG. 7, a first conductive ink layer 75 is printed on the substrate 30, and more specifically, on the sensor section 38 thereof. A piezoelectric ink layer 76 is printed on top of first conductive ink layer. For example, the piezoelectric ink layer may be polyvinylidene difluoride (PVDF). A second conductive ink layer 77 is printed over the piezoelectric ink layer 76. The first and second conductive ink layers 75 and 77 may be the same conductive ink as the stimulation circuit 50 and the leadwires 51, 52, 64, 65, such as the silver-based first conductive ink described above. In such an embodiment, the charge produced by the piezoelectric ink layer 76 from the motion or bend can be measured as a produced voltage between the first conductive ink layer 75 and the second conductive ink layer 77, which act as electrodes.

A dielectric ink layer 78 is printed or otherwise applied over the second conductive ink layer 77 to isolate and protect the conductive ink layer. In certain examples, the dielectric layer 78 may be printed over just the second conductive ink layer 77 or may be printed over the entire sensor section 38, and/or over the first and/or second lead sections 35 and 37 of the substrate 30 as well.

The layer configuration depicted in FIG. 7 exemplifies just one configuration of the piezoelectric bend sensor 62b. Other configurations may have more or fewer layers depending on the requirements of the measurement system. For example, increasing the number of piezoelectric layers may enable collection and generation of a greater charge magnitude resulting from the muscle response.

FIGS. 4 and 5 depict the printed bend sensor 62 as having a serpentine configuration on the sensor section 38 of the substrate 30. Other shapes or patterns may be used for the printed bend sensor 62 such as a spiral or any other pattern that increases the length of conductor exposed to the bending.

In some embodiments, an adhesive layer may be applied over the top of the printed bend sensor 62 to adhere the printed bend sensor to the support frame 22, for example. FIGS. 6 and 7 depict each of the resistive sensor 62a and the piezoelectric sensor 62b having an adhesive layer configured to adhere the respective sensor to the support frame 22, and particularly to the bendable middle section 24 thereof. In FIG. 6, the adhesive layer 73 is applied over the dielectric ink layer 72, and thus over the resistive sensor 62a. In FIG. 7, the adhesive layer 79 is applied over the dielectric ink layer 78. The adhesive layers 73, 79 may be printed over the entirety of the sensor section 38. The adhesive layers 73, 79 may be printed adhesive or may be otherwise be rolled or sprayed over the top of the printed bend sensor 62 and/or the sensor section 38. To provide one example, the adhesive layer 73, 79 may be double-sided pressure sensitive adhesive tape with acrylic adhesive.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A kinemyography sensor comprising:
a support frame configured to attach to a patient's thumb and forefinger and having a bendable middle section configured to bend in response to movement of the patient's thumb;
a flexible substrate, wherein at least a portion of the flexible substrate is attached to the support frame;
a printed stimulation circuit printed on the substrate and comprising a pair of stimulation electrodes configured to adhere to a patient's skin to deliver a kinemyography stimulus; and
a printed bend sensor printed on the substrate and located on the bendable middle section of the support frame, wherein the printed bend sensor is configured to sense the bending of the support frame.

2. The kinemyography sensor of claim 1, wherein the printed bend sensor comprises a printed resistive sensor configured to change resistance when the bendable middle section bends so as to sense the movement of the patient's thumb.

3. The kinemyography sensor of claim 2, wherein the printed resistive sensor includes a conductive ink layer printed on the substrate and a dielectric ink layer printed on the conductive ink layer.

4. The kinemyography sensor of claim 1, wherein the printed bend sensor comprises a printed piezoelectric sensor configured to produce a charge when the bendable middle section bends so as to sense the movement of the patient's thumb.

5. The kinemyography sensor of claim 4, wherein the printed piezoelectric sensor includes:
a first conductive ink layer printed on the substrate;
a piezoelectric ink layer on the first conductive ink layer;
a second conductive ink layer printed on the piezoelectric ink layer; and
a dielectric ink layer on the conductive ink layer.

6. The kinemyography sensor of claim 1, flexible substrate includes an elongated body with a first end and a second end, wherein the elongated body includes:
a connection section at the first end having a plurality of contact pads printed thereon and configured to mate with a sensor connector of a neuromuscular transmission monitoring device;
a sensor section having the printed bend sensor is printed thereon, wherein the sensor section is attached to the bendable middle section of the support frame; and
a stimulation section having the pair of stimulation electrodes printed thereon.

7. The kinemyography sensor of claim 6, wherein the sensor section is at the second end of the flexible substrate.

8. The kinemyography sensor of claim 6, wherein the stimulation section is at the second end of the flexible substrate.

9. The kinemyography sensor of claim 6, wherein the elongated body further includes:
a first lead section between the connection section and the stimulation section, the first lead section having at least two stimulation leadwires and at least two sensor leadwires printed thereon; and
a second lead section between the stimulation section and the sensor section, the second lead section having the two sensing leads printed thereon.

10. The kinemyography sensor of claim 6, wherein the plurality of contact pads printed on the connection section includes at least two sensing contact pads and two stimulation contact pads, wherein the two sensing contact pads are each connected to a sensing leadwire and the two stimulation contact pads are each connected to a stimulation leadwire.

11. The kinemyography sensor of claim 1, wherein the support frame is a molded polymer having curved shape with a first leg configured to attach to the patient's thumb and a second leg configured to attach to the patient's forefinger.

12. A kinemyography sensor comprising:
a flexible substrate including:
a stimulation section having a pair of stimulation electrodes printed thereon and configured to adhere to a patient's skin to deliver a kinemyography stimulus;
a sensor section having a printed bend sensor printed thereon, wherein the printed bend sensor is configured to be positioned between a patient's thumb and forefinger to sense movement of the patient's thumb; and
a connection section at a first end of the substrate, the connection section having a plurality of contact pads printed thereon and configured to mate with a sensor connector of a neuromuscular transmission monitoring device.

13. The kinemyography sensor of claim 12, wherein the printed bend sensor comprises a printed resistive sensor configured to change resistance when bent.

14. The kinemyography sensor of claim 12, wherein the printed bend sensor comprises a printed piezoelectric sensor configured to produce a charge when the bendable middle section bends.

15. The kinemyography sensor of claim 12, wherein the flexible substrate further includes:
two printed stimulation leadwires, one extending from each of the pair of stimulation electrodes to a respective stimulation contact pad on the connection section; and
two printed sensor leadwires, one extending from each end of the printed bend sensor to a respective sensing contact pad on the connection section.

16. The kinemyography sensor of claim 15, wherein the flexible substrate further includes:
a first lead section between the connection section and the stimulation section, the first lead section having the stimulation leadwires and the sensor leadwires printed thereon; and
a second lead section between the stimulation section and the sensor section, the second lead section having the two sensing leads printed thereon.

17. The kinemyography sensor of claim 15, wherein the connection section includes the sensing contact pads and stimulation contact pads printed thereon.

18. The kinemyography sensor of claim 15, wherein the sensor section is at a second end of the flexible substrate.

19. The kinemyography sensor of claim 12, wherein the stimulation section is at a second end of the flexible substrate.

20. The kinemyography sensor of claim 12, further comprising a support frame configured to attach to a patient's thumb and forefinger and having a bendable middle section configured to bend in response to movement of the patient's thumb, wherein the sensor section is attached to the bendable middle section.

21. A neuromuscular transmission monitoring system comprising:
a kinemyography sensor;
a neuromuscular transmission monitoring device including a sensor connector configured to removably mate with a first end of the kinemyography sensor so as to receive sensing signals therefrom;
wherein the kinemyography sensor comprises a flexible substrate including:
a stimulation section having a pair of stimulation electrodes printed thereon and configured to adhere to a patient's skin to deliver a kinemyography stimulus;
a sensor section comprising a printed bend sensor printed thereon and configured to sense movement of the patient's thumb in response to the stimulus, wherein the printed bend sensor is a resistive sensor or a piezoelectric sensor; and
a connection section at the first end of the substrate and configured to electrically connect to the sensor connector.

22. The system of claim 21, wherein the kinemyography sensor further comprises a support frame configured to attach to a patient's thumb and forefinger and having a bendable middle section configured to bend in response to movement of the patient's thumb, wherein the sensor section of the flexible substrate is attached to the bendable middle section.
